(19) United States
(12) Patent Application Publication
(10) Pub. No.: US 2024/0398912 A1
(43) Pub. Date: Dec. 5, 2024

(54) METHOD OF MITIGATION OF INJURIES CAUSED BY SYSTEMIC GENOTOXIC STRESS

(71) Applicant: Health Research, Inc., Buffalo, NY (US)

(72) Inventors: Andrei GUDKOV, East Aurora, NY (US); Craig BRACKETT, Hamburg, NY (US)

(21) Appl. No.: 18/691,035

(22) PCT Filed: Sep. 22, 2022
(86) PCT No.: PCT/US2022/076879

§ 371 Date: Mar. 11, 2024

Related U.S. Application Data
(60) Provisional application No. 63/247,116, filed on Sep. 22, 2021.

(51) Int. Cl. A61K 38/48; A61P 39/00

(52) U.S. Cl. CPC A61K 38/4886; A61P 39/00; C12Y 304/24035

(57) ABSTRACT

Provided are methods for therapy or prophylaxis of genotoxic stress. The methods include administering to an individual in need an effective amount of Matrix Metalloproteinase (MMP)-9. The individual in need may have or be at risk of developing Acute Radiation Syndrome (ARS), or may have received or is receiving chemotherapy, or has insufficient hematopoietic function. The present disclosure provides results from a mouse model of lethal ARS induced by TBI to demonstrate that neutrophils (N) are essential mediators of the radiomitigative but not radioprotective abilities of entolimod, express functional TLR5 butundergo minimal transcriptional changes post-entolimod suggesting that N mitigate 30 ARS through a transcriptional-independent mechanism; and increase the number of active hematopoietic B pluripotent precursors (HPPs) in bone marrow.

### CROSS REFERENCE TO RELATED APPLICATIONS

This application claims priority to U.S. provisional application No. 63/247,116, filed Sep. 22, 2021, the entire disclosure of which is incorporated herein by reference.

### FIELD

The present disclosure relates generally to use of matrix metalloproteinase-9 (MMP-9) for prophylaxis or treatment of cell damage caused by genotoxic agents such as ionizing radiation and chemotherapeutic drugs.

### SEQUENCE LISTING

The instant application contains a Sequence Listing which has been submitted in .xml format and is hereby incorporated by reference in its entirety. Said .xml copy was created on Sep. 22, 2022, is named “003551_01057_ST26.xml”, and is 2,495 bytes in size.

### RELATED INFORMATION

Exposure to ionizing radiation causes significant DNA damage in many cells of the organism and is the underlying cause of multiple pathologies including acute radiation sickness (ARS). They can occur either accidentally (i.e., nuclear disasters like Chernobyl of Fukushima catastrophes) or intentionally (i.e., cancer therapies) and require the development of countermeasures that can reduce the severity of ARS and improve recovery and survival. Such countermeasures are classified as either protectants when given prophylactically prior to intentional radiation or mitigators when administered therapeutically after radiation exposure [1].

Development of protectors has been more successful than radiomitigators. The only FDA-approved powerful radioprotective drug, an antioxidant amifostine, has no efficacy as a radiomitigator [2]. In fact, dramatic cell loss in the hematopoietic system (HPS) due to p53-mediated apoptosis induced by DNA damage occurs within the first few hours after radiation exposure [3, 4] and renders radioprotective agents completely ineffective. Similarly, agents that induce proinflammatory cytokines (e.g., TNF, IL-12) [5, 6] are another well-studied class of efficacious radioprotectants that commonly act as activators of a pro-survival p53-suppressive NF-κB pathway [7].

Efficacious radiomitigators engage mechanisms of tissue regeneration and reduce the risk of sepsis and bleeding rather than suppressing apoptosis [8]. The only class of FDA-approved drugs for such indications are derivatives of G-CSF (Neupogen and Neulasta) commonly used in oncology to accelerate the recovery of hematopoiesis following chemotherapy-associated myelosuppression [1]. Unfortunately, efficacy of these G-CSF-based drugs is rather weak, and they require multiple administrations under conditions of supportive clinical care [9-11] and do not satisfy the needs of mass casualty scenarios. Thus, the development of potent yet feasible mitigating countermeasures remains a strong unmet medical need.

Bacterial flagellin, the sole toll-like receptor (TLR) 5 agonist, has been identified as a superior radioprotectant when compared to amifostine for suppressing apoptosis in radiosensitive tissues [12]. A pharmacologically optimized flagellin derivative, entolimod, has been developed as a powerful radioprotector in mice and non-human primates but, unlike amifostine, did not diminish the radiosensitivity of tumors [12]. Moreover, entolimod appeared to be an effective radiomitigator in rodent and non-human primate models of lethal ARS caused by total body irradiation (TBI). A single dose of entolimod with no supportive care rescued animals when administered within 48 hours post-TBI [13, 14] thus exceeding the efficacy of G-CSF-based drugs. Remarkably, entolimod also showed efficacy in other pre-clinical models of both genotoxic [15-17] and non-genotoxic stressors [18-20].

While entolimod-induced radioprotection is well-known to involve a combination of anti-apoptotic, anti-oxidative and anti-bacterial effects elicited by the liver [12, 13], the mechanism of radiomitigation remains poorly understood. There is therefore an ongoing need to develop new agents for addressing genotoxic stress caused by ionizing radiation and other agents that damage DNA. The present disclosure is pertinent to this need.

### BRIEF SUMMARY OF DISCLOSURE

The present disclosure provides results from a mouse model of lethal ARS induced by TBI to demonstrate that neutrophils (Nϕ) (i) are essential mediators of the radiomitigative but not radioprotective abilities of entolimod, (ii) express functional TLR5 but undergo minimal transcriptional changes post-entolimod suggesting that Nϕ mitigate ARS through a transcriptional-independent mechanism; and (iii) increase the number of active hematopoietic pluripotent precursors (HPPs) in bone marrow. The disclosure reveals these effects can be mimicked by administration of recombinant pro-MMP-9 (rMMP-9) in the absence of Nϕ. These results define the release of MMP-9 by Nϕ as a major contributor, along with G-CSF and IL-6 [14], as mediators of radiomitigation post-entolimod by increasing the number of active HPPs to facilitate recovery of a damaged HPS. These data support the use of MMP-9 to compensate for the shortcomings of currently approved radiation countermeasures, and for use in treating or preventing other conditions that relate to DNA damage, particularly in hematopoietic cells, and for prophylaxis or treatment of hematological disorders associated with insufficient hematopoietic cells.

### BRIEF DESCRIPTION OF THE FIGURES

FIG. 1. Nϕ mediate the radiomitigative but not radioprotective abilities of entolimod. Experimental layouts to determine the role of Nϕ in the (A) radioprotective and (E) radiomitigative effects of entolimod in BALB/c mice. Survival by Kaplan-Meier curves in mice treated with entolimod in the presence (rat IgG) or absence (a-Ly-6G) of Nϕ in the (B) radioprotective and (F) radiomitigative schemes. P-values were determined by Log-rank test. Measurement of total HPPs and granulocyte/macrophage (G/M) progenitors in BM by MethoCult in the (C) radioprotective (n=4-5 mice/group) and (G) radiomitigative (n =3-10 mice/group) schemes. Total HPPs were measured ⁺1 h post-TBI for radioprotection and for radiomitigation, both total HPPs and G/M progenitors ⁺3d post-treatment with vehicle or entolimod. (D) Absolute number of stem cell populations in BM by flow cytometry in the presence (rat IgG) and absence (a-Ly-6G) of N on day 8 post-TBI in the radioprotection scheme (n=3-5 mice/group). Stem cell populations were defined as follows: HSC (Lineage⁻ Flt3⁻ c-kit⁺ Sca-1⁺), MMP (Lineage⁻ Flt3⁺c-kit⁺ Sca-1⁺), CLP (Lineage⁻ Flt3 ⁻IL-7R⁺ c-kit⁺ Sca-1⁺), Myeloid (Lineage⁻ Flt3⁻ IL-7R⁻ c-kit⁻ Sca-1⁻), CMP (Lineage⁻ Flt3⁻ IL-7R⁻ CD34⁺CD16/32⁻), MEP (Lineage⁻ Flt3⁻ IL-7R⁻ CD34⁻ CD16/32⁻), and GMP (Lineage-Flt3⁻ IL-7R⁻ CD34⁺CD16/32⁺). Error bars represent mean ±SEM; P-values were determined by Student's t test.

FIG. 2. Entolimod stimulates Nϕ recruitment to tissues and differentiation. (A) Fold change in the Nϕ response in the indicated organs calculated from the absolute number of Nϕ in entolimod treated (2 h and 5 h) versus vehicle treated mice (n=3-5 mice/group). Flow cytometry was used to identify Nϕ (CD45⁺CD11b⁺Ly-6C⁺Ly-6G^{h}). (B) Absolute number of endogenous and adoptively transferred Nϕ in the BM, liver, and lungs 2 h post-treatment with vehicle or entolimod (n=6-8 mice/group). Adoptive transfers were done by i.v. injection of GFP-expressing BM immediately before treatment. Endogenous versus transferred Nϕ were distinguished by flow cytometry for GFP among the total Nϕ population (CD45⁺CD11b⁺Ly-6C⁺Ly-6G). (C) Absolute number (left) and composition (right) of Nϕ populations in the bone marrow at the indicated time points post-treatment by flow cytometry as defined by: pre-Nϕ(Lineage⁻ c-kit^{int} CD115⁻ CD11b⁺Gr-1⁺CXCR4⁺), immature Nϕ (Lineage⁻ c-kit^{int} CD115⁻ CD11b⁺Gr-1⁺CXCR4⁻ CXCR2⁻ Ly-6G⁺), and mature Nϕ (Lineage⁻ c-kit^{int} CD115⁻ CD11b⁺Gr-1⁺CXCR4⁻ CXCR2⁺Ly-6G⁺) where lineage was defined as CD3⁻ and B220⁻ (n=3-9 mice/group). (D) Flow cytometry based staining for TLR5 using biotinylated-entolimod plus streptavidin fluorochrome “sandwich” platform on Nϕ (CD45⁺CD11b⁺Ly-6C⁺Ly-6G^{hi}), NK cells (CD45⁺CD3E⁻ NKp46⁺), DCs (CD45⁺Ly-6G⁻ B220⁻ F4/80⁻ CD11c⁺), monocytes (CD45⁺Ly-6G⁻ B220⁻ CD11c⁻ F4/80⁺), B cells (CD45⁺Ly-6G⁻ CD11c⁻ F4/80⁻ B220⁺), CD8⁺T cells (CD45⁺CD3ε⁺CD8⁺), CD4⁺T cells (CD45⁺CD3, ⁺CD4⁺), platelets (CD45⁻ CD41⁺), and RBCs (CD45⁻ Ter 19⁺) in the blood of BALB/c and Tlr5^{-/-} mice. Error bars represent mean ±SEM. *P<0.04; P-values were determined by Student's t test.

FIG. 3. TLR5 activation on Nϕ and non-hematopoietic cells cooperate to mitigate radiation damage. Tlr5^{-/-} bone marrow chimeric mice received an adoptive transfer of either Tlr5^{WT/WT} or Tlr5^{-/-} Nϕ (10 million/mouse) followed immediately by vehicle or entolimod 24 h post-TBI. (A-B) Survival was measured by Kaplan-Meier curves. P-values were determined by Log-rank test; n=10-16 mice/group. (C) Measurement of total HPPs by MethoCult in BM on day 7 post-treatment using the same experimental setup as (A). Error bars represent mean ±SEM; P-values were determined by Student's t test; n=4-7 mice/group. In all instances Nϕ purity was routinely greater than 98%.

FIG. 4. Entolimod stimulates Nϕ to release MMP-9. Protein microarray analysis of (A) mouse serum using R&D Systems Proteome Profiler Mouse XL Cytokine Array in the presence (rat IgG) and absence (a-Ly-6G) of N 24 h post-TBI and (B) Nϕ isolated from BM that were ex vivo treated with vehicle or entolimod for 30 minutes. Measurement by ELISA of MMP-9 in (C) serum from mice (n=5/group) in the presence (rat IgG) and absence (α-Ly-6G) of N 2 h post-treatment with vehicle or entolimod. Mice received TBI 24 h prior to vehicle or entolimod treatment and (D) supernatants from Tlr5^{WT/WT} and Tlr5^{-/-} Nϕ isolated from BM (n=6 mice/group) that were ex vivo treated with vehicle or entolimod for 30 minutes.

FIG. 5. Release of MMP-9 by Nϕ post-entolimod mitigates lethal radiation damage to hematopoiesis. (A) BALB/c mice (n=5/group) were given 5 Gy TBI followed 24 h later by treatment with vehicle, entolimod, or the indicated doses of recombinant pro-MMP-9 (rMMP-9). Measurement of total HPPs in BM by MethoCult was done on day 3 post-treatment. (B) Survival by Kaplan-Meier curve in BALB/c mice treated with vehicle, entolimod, or 10 μg/kg rMMP-9 24 h post 7.5 Gy TBI (n=9-10 mice/group). P-values were determined by Log-rank test. (C) BALB/c mice (n=5/group) were given 5 Gy TBI followed 6 h later by rat IgG or α-Ly-6G. Twenty-four hours later mice were treated with vehicle, entolimod, rMMP-9 (10 pg/kg), or rMMP-9 plus entolimod. Measurement of total HPPs in BM by MethoCult was done on day 3 post-treatment. For (A) and (C) error bars represent mean ±SEM; P-values were determined by Student's t test.

FIG. 6. Proposed mechanism by which entolimod mitigates lethal radiation damage to hematopoiesis.

FIG. 7. Effective depletion of Nϕ with α-Ly-6G antibody. The experimental design outlined in FIG. 1E was used. Nϕ were measured in blood of mice given rat IgG or α-Ly-6G by complete blood cell count with differential 2 h post-treatment with vehicle or entolimod. Error bars represent mean ±SEM; P-values were determined by Student's t test; n=5 mice/group.

FIG. 8. Nϕ do not mediate the radioprotective activities of entolimod in outbred NIH-S mice. The experimental design outlined in FIG. 1A was used. (A) Survival by Kaplan-Meier curves in mice treated with entolimod in the presence (rat IgG) or absence (α-Ly-6G) of Nϕ. P-values were determined by Log-rank test; *P<0.001. (B) Measurement of total HPPs by MethoCult (n=4-5 mice/group) and (C) absolute number of stem cell populations by flow cytometry in the presence (rat IgG) or absence (α-Ly-6G) of N on day 8 post-treatment with vehicle or entolimod (n=4-5 mice/group). Error bars represent mean SEM; P-values were determined by Student's t test.

FIG. 9. Entolimod increases the trafficking properties of N*. (A) Nϕ magnetically isolated from the bone marrow of Tlr5^{WT/WT} mice were treated ex vivo with vehicle or entolimod (8 pM) for the indicated time points and stained for flow cytometry with mAb against L-selectin and CD1 lb. Percent L-selectin down-regulation was calculated using the formula [(MFI_{entolimod}/MFI_{vehicle})-1]×100%. Percent CD11b up-regulation was calculated using the formula (MFI_{entolimod}/MFI_{vehicle})×100%; n=3 mice/group. (B) Nϕ magnetically isolated from the bone marrow of Myd88^{WT/WT} and Myd8^{-/-} mice were treated ex vivo with vehicle or entolimod (8 pM) for the indicated time points and stained for flow cytometry with mAb against L-selectin and CD11b. L-selectin down-regulation and CD11b up-regulation were calculated as in (A); n=3 mice/group. Blood from Myd88^{WT/WT} Myd88^{-/-}, and Tlr5^{-/-} mice was RBC lysed and stained with our b-entolimod flow cytometry “sandwich” platform to measure TLR5 expression on Nϕ (CD45⁺CD11b⁺Ly-6C^{lo} Ly-6G^{hi}). (C) Nϕ isolated from the bone marrow of Tlr5^{WT/WT} mice were fluorescently labeled with 10 μM CFDA. Endothelial cells (2H-11) were either left untreated or treated for 30 minutes with vehicle or entolimod (3 pM), washed once, and incubated for another 4.5 h prior to the addition of CFDA-labeled Nϕ. Fluorescence was measured after washing four times. Percent Nϕ binding was calculated using the formula (Fluorescence_{entolimod}/Fluorescence_{vehicle})×100% after which background fluorescence was subtracted from vehicle and entolimod treated fluorescence values; n=3 mice/group. (D) L-selectin and CD11b mediated recruitment of Nϕ (CD45⁺CD11b⁺ Ly-6C⁺Ly-6G^{hi}) to the liver 2 h post-entolimod. Cohorts of mice were given blocking antibodies to L-selectin or ICAM-1 or appropriate isotype controls 30 minutes prior to entolimod. Fold change was calculated based on the absolute number of N in mice treated with entolimod versus vehicle for each antibody treatment group; n=4-8 mice/group. Error bars represent mean ±SEM and p-values were determined by Student's t test.

FIG. 10. The radiomitigative activity of entolimod is TLR5-dependent. (A) Survival by Kaplan-Meier curve in Tlr5^{WT/WT} and Tlr5^{-/-} mice treated with vehicle or entolimod 24 h post-TBI. P-values were determined by Log-rank test; *P<0.04 when comparing Tlr5^{WT/WT} and #P<0.007 when comparing Tlr5^{WT/WT} Tlr5^{-/-}. (B) Measurement of total HPPs by MethoCult from the BM of Tlr5^{WT/WT} and Tlr5^{-/-} mice on day 7 post-treatment. Cohorts of mice given TBI were treated with vehicle or entolimod 24 h later and in the case of the no TBI cohort mice were treated in a similar manner (n=6-10 mice/group). Error bars represent mean ±SEM; P-values were determined by Student's t test.

FIG. 11. TLR5 stimulation on non-hematopoietic cells releases G-CSF and IL-6. Measurement of serum levels of G-CSF and IL-6 by ELISA in (A) the presence (rat IgG) and absence (α-Ly-6G) of N (n=10 mice/group) or (B) Tlr5^{-/-} bone marrow chimeric mice 2 h post-treatment with vehicle or entolimod (n=4-6 mice/group). Mice received TBI 24 h prior to vehicle or entolimod treatment. Error bars represent mean ±SEM; P-values were determined by Student's t test.

FIG. 12. Entolimod causes minimal transcriptional changes in N<. Volcano plots showing differentially expressed genes in (A) Nϕ (n=2 mice/group) and (B) liver (n=3 mice/group) with dots showing genes with statistically significant differential expression. Dashed line show 0 adj<0.05 significance threshold; for visualization purposes, all p-values_{adj} below le-15 were assigned this level (shown as dotted line). Below the volcano plots are histograms of distribution of log 2(FC) in statistically significant differential expressed genes in Nϕ and liver. (C) Venn diagrams for list of genes which are up-regulated (p-value_{adj}<0.05, log 2(FC)>1; top) or down-regulated (p-value_{adj}<0.05, log 2(FC)<−1; bottom) in Nϕ and liver and various dots show intersection. (D) Scatter plot for per-gene log 2(FC) in Nϕ and liver. Dots show genes which are DE with p-value_{adj}<0.05 in Nϕ only, in liver only, and genes which are DE with p-value_{adj}<0.05 both in Nϕ and liver. Dashed lines show log 2(FC)=−1/1 thresholds; dash-and-dotted lines show thresholds of equal absolute log 2(FC) values. (E) Fold change in NF-κB driven luciferase activity in Nϕ (n =4-6 mice/group) and liver (n=5 mice) post-entolimod. Ex vivo indicates that Nϕ were treated with vehicle or entolimod for 3 h after isolation prior to luciferase measurement. In vivo indicates that mice were treated with vehicle or entolimod for 3 h followed by Nϕ isolation and immediate luciferase measurement. For liver, luciferase activity was measured in protein lysates collected from mice 3 h post-treatment with vehicle or entolimod. Error bars represent mean ±SEM.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide and amino acid sequences described herein. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included, as are derivatives thereof. The disclosure includes all polynucleotide and all amino acid sequences that are identified herein by way of a database entry. Such sequences are incorporated herein by reference as they exist in the database on the filing date of this application or patent.

In embodiments, the disclosure provides for administering MMP-9 or a biologically active derivative thereof to an individual in need. The disclosure also includes ex vivo cell populations that have been contacted with exogenously provided MMP-9 or a biologically active derivative thereof. The compositions and methods are suitable for use with humans, and non-human mammals. Thus, the disclosure encompasses human and veterinary uses.

In embodiments, the disclosure provides for use of MMP-9 having an amino acid sequence that is the same as, or is at least 80% similar, to the amino acid sequence of any a MMP-9 produced by a mammal, such as human, non-human primate, canine, feline, equine, or porcine mammal. In a non-limiting embodiment, the MMP-9 comprises the amino acid sequence of human MMP-9, which is known in the art. In embodiments, the MMP-9 comprises an MMP-9 pre-protein, or a processed protein. In a non-limiting embodiment, the MMP-9 amino acid sequence comprises or consists of the sequence:

(SEQ ID NO: 1)
MSLWQPLVLVLLVLGCCFAAPRQRQSTLVLFPGDLRTNLTDRQLAEEYLY
RYGYTRVAEMRGESKSLGPALLLLQKQLSLPETGELDSATLKAMRTPRCG
VPDLGRFQTFEGDLKWHHHNITYWIONYSEDLPRAVIDDAFARAFALWSA
VTPLTFTRVYSRDADIVIQFGVAEHGDGYPFDGKDGLLAHAFPPGPGIQG
DAHFDDDELWSLGKGVVVPTRFGNADGAACHFPFIFEGRSYSACTTDGRS
DGLPWCSTTANYDTDDRFGFCPSERLYTQDGNADGKPCQFPFIFQGQSYS
ACTTDGRSDGYRWCATTANYDRDKLFGFCPTRADSTVMGGNSAGELCVFP
FTFLGKEYSTCTSEGRGDGRLWCATTSNFDSDKKWGFCPDQGYSLFLVAA
HEFGHALGLDHSSVPEALMYPMYRFTEGPPLHKDDVNGIRHLYGPRPEPE
PRPPTTTTPQPTAPPTVCPTGPPTVHPSERPTAGPTGPPSAGPTGPPTAG
PSTATTVPLSPVDDACNVNIFDAIAEIGNQLYLFKDGKYWRFSEGRGSRP
QGPFLIADKWPALPRKLDSVFEERLSKKLFFFSGRQVWVYTGASVLGPRR
LDKLGLGADVAQVTGALRSGRGKMLLFSGRRLWRFDVKAQMVDPRSASEV
DRMFPGVPLDTHDVFQYREKAYFCQDRFYWRVSSRSELNQVDQVGYVTYD
ILQCPED

The disclosure includes derivatives of the MMP-9 amino acid sequence. Derivatives include, for example, N-terminal or C-terminal truncations, amino acid substitutions. In embodiments, an MMP-9 sequence comprises 10-707 contiguous amino acids of SEQ ID NO: 1, inclusive, and including all numbers and ranges of numbers between 100 and 707. In embodiments, a catalytically active segment of the MMP-9 protein is used. In embodiments, the MMP-9 derivative comprises one or more conservative amino acid substitutions. In embodiments, the MMP-9 derivative comprises an MMP-9 amino acid sequence as a component of a fusion protein. In an embodiment, the MMP-9 protein is provided in a contiguous polypeptide with additional amino acids that are intended to, for example, improve expression, facilitate purification (e.g., any of a variety of known purification tags), extend half-life, and/or improve bioavailability. In a non-limiting embodiment, the MMP-9 protein is present in a contiguous polypeptide with an Fc region of an immunoglobulin. In embodiments, the MMP-9 protein is produced recombinantly. Thus, the MMP-9 protein can be expressed from an expression vector encoding a described MMP-9 protein that is introduced into a cell culture such that cells that comprise the vector express the protein. The MMP-9 protein can be isolated from the cells using well known techniques and purified to any desired degree of purity. Alternatively, the MMP-9 protein may be isolated from cells that produce it normally without genetic engineering.

In embodiments, an effective amount of a described MMP-9 is administered to an individual in need thereof. An effective amount means an amount of protein that will elicit the biological or medical response by a subject that is being sought by a medical doctor or other clinician. In embodiments, an effective amount means an amount sufficient to prevent, or reduce by at least about 30 percent, or by at least 50 percent, or by at least 90 percent, any sign or symptom of ARS, or other genotoxic injury. In embodiments, an effective amount comprises an amount of a described MMP-9 protein that is sufficient to stimulate proliferation of hematopoietic stem cells. In embodiments, the hematopoietic stem cells that are dormant prior to being contacted with the MMP-9 protein. In embodiments, a single dose of a described MMP-9 protein is sufficient to function as a radioprotectant or a radiomitigator.

In embodiments, the described MMP-9 proteins can be provided as pharmaceutical formulations. A pharmaceutical formulation can be prepared by mixing the polypeptides with any suitable pharmaceutical additive, buffer, and the like. Examples of pharmaceutically acceptable carriers, excipients and stabilizers can be found, for example, in Remington: The Science and Practice of Pharmacy (2012) 22nd Edition, Philadelphia, PA. Lippincott Williams & Wilkins, the disclosure of which is incorporated herein by reference.

In embodiments, a described MMP-9 protein is administered to an individual in need thereof. In embodiments, the individual in need is at risk of being exposed to DNA damaging amounts or ionizing radiation. In embodiments, the ionizing radiation is due to, for example, a radiation disaster, or intentional exposure to ionizing radiation, such as during a medical procedure. In embodiments, the individual has been exposed to DNA damaging amounts of ionizing radiation. In embodiments, use of the described MMP-9 protein produces a beneficial effect without promoting an IL-6-driven inflammatory response.

In embodiments, the individual is need of prophylaxis or therapy for a condition associated with insufficient hematopoiesis. In embodiments, insufficient hematopoiesis comprises fewer erythroid precursor cells in bone marrow than erythroid precursor cells in bone marrow in an individual who does not have insufficient hematopoiesis, e.g., an individual who has normal bone marrow. In embodiments, insufficient hematopoiesis may be evidenced by decreased numbers of mature, functioning blood cells in circulation. In embodiments, the individual has or is at risk of developing any type of anemia, including aplastic anemia, or a myelodysplastic syndrome, or the like. In embodiments, the individual has received or is being prepared to receive a bone marrow transplant.

In embodiments, the individual is a candidate to receive an agent that my promote, promotes systemic genotoxic stress (SGS). In embodiments, SGS is a DNA damaging agent. In embodiments, the individual to which a described MMP-9 protein is administered is a candidate to receive an SGS inducing agent, or is receiving the SGS inducing agent. In embodiments, the agent is a DNA damaging chemotherapeutic agent. Non-limiting examples of chemotherapeutic agents for which MMP-9 administration may provide a beneficial effect include but are not limited to intercalaters, DNA alkylators such as cyclophosphamide, chlorambucil, and melphalan, alkylating-like platinum agents such as cisplatin and cisplatin analogs, carboplatin, or other agents, such as Methotrexate, Doxorubicin, Daunorubicin, pyrimidine analogs and purine analogs, or mitomycin C. Thus, in embodiments, the described compositions and methods are used for improving outcomes for cancer patients who receive chemotherapy, radiation, or a combination thereof.

The disclosure includes isolated populations of cells, such as hematopoietic stem cells, that have been contacted with an exogenous MMP-9 protein. In embodiments, an autologous population of cells that includes hematopoietic stem cells is exposed to an exogenous MMP-9 protein to, for example, promote hematopoiesis after implantation to a recipient.

Use of the MMP-9 protein as a single agent to achieve any of the described effects is included within the scope of the disclosure. However, the disclosure includes in certain embodiments combinations of agents that include MMP-9. The additional agents may be radioprotectants, radiomitigants, anti-oxidants, anti-inflammatory agents, and the like. In embodiments, the disclosure includes administering to an individual in need a combination of the described MMP-9 proteins or derivatives thereof with granulocyte colony-stimulating factor (GCSF)-based drugs, non-limiting examples of which include Filgrastim, sold under the brand name NEUPOGEN and Pegfilgrastim sold under the brand name NEULASTA.

The following Examples are intended to illustrate but not limit the disclosure.

### Material and Methods

Mice. Pathogen-free inbred BALB/c mice were obtained from The Jackson Laboratories or from our colony maintained at Roswell Park. BALB/c-Tlr5^{-/-} mice obtained from Dr. Shibata [22] and C57BL/6-GFP breeders [C57BL/6-Tg(UBC-GFP)30Scha/J]purchased from The Jackson Laboratories were all bred and maintained by Roswell Park Laboratory Animal Shared Resource (LASR). All mice were housed in microisolator cages in a laminar flow unit under ambient light. The Roswell Park Institutional Animal Care and Use Committee (IACUC) approved all procedures carried out in this study. Adult female mice between the ages of 11 and 12 weeks were assigned randomly to groups; group sizes were selected based on prior experience. No animals were excluded from further analysis in the reported studies.

Generation of Tlr5^{-/-} bone marrow chimeric mice. BALB/c and Tlr5^{-/-} mice received a fractionated dose (3.75 Gy 3 h apart) of TBI using a 4000 Ci Cesium-137 source (J. L. Shepherd and Associates, San Fernando, CA) on a rotating platform to ensure even dose delivery to all tissues. Mice received syngeneic bone marrow transplantation by intravenous (i.v.) injection 24 h later. Mice rested for at least 9 weeks prior to enrollment into studies.

Reagents. The TLR5 agonist entolimod (CBLB502) is a cGMP-manufactured drug product that was obtained from Cleveland BioLabs, Inc. [12, 23]. We purchased mouse MethoCult GF and MethoCult without cytokines from STEMCELL Technologies, recombinant mouse G-CSF from Peprotech, rat IgG2a, κ(clone 2A3) and α-Ly-6G (clone 1A8) from BioXcell, Proteome Profiler Mouse XL Cytokine Array from R&D Systems, Duoset ELISA kits from R&D Systems, and recombinant mouse MMP-9 from BioLegend. All reagents were handled according to the manufacturer's instructions.

Radioprotection and radiomitigation studies. Mice received 7.5 Gy TBI similarly as described above for all studies except for non-survival radiomitigation studies in which mice received 5 Gy TBI. Mice received vehicle (PBS) or entolimod (1 pg) s.c. 30 minutes pre-TBI for radioprotection studies [12, 15, 19] or 24 h post-TBI for radiomitigation studies [13, 14].

Nϕ depletion and isolation. To effectively deplete Nϕ, mice were intraperitoneally (i.p.) administered mAbs against Ly6G (100 pg/mouse) 18 h prior to vehicle or entolimod administration. Isotype-matched controls (rat IgG2a, x) were given at the same dose and regimen as the depletion mAbs. Nϕ isolation and transfer were achieved by magnetic bead isolation from the bone marrow of naive mice according to the manufacturer's protocol using the ultrapure Ly6G microbead kit (Miltenyi Biotec). Purity was routinely greater than 98%. Nϕ were transferred via i.v. injection immediately before vehicle or entolimod administration.

FACS staining and analysis. Single cell suspensions of tissues were generated as outlined (Table 1). Erythrocytes were lysed using a solution of ammonium chloride for 5 minutes at room temperature except for when Ter-119 was used in Panel 1. Live cells were counted by trypan blue exclusion, re-suspended in phosphate azide buffer (PAB), and stained on ice for 10 minutes with TruStain fcX™ (clone 93, BioLegend for Panels 1 and 2) or rat serum (for Panel 3) followed by a cocktail of mAbs or isotype-matched controls for 15 minutes (Table 2). We biotinylated entolimod with the EZ-Link™ Sulfo-NHS-LC-Biotinylation Kit (ThermoFisher). After staining, cells were washed once with PAB and fixed in 2% ultrapure formalin (Polysciences). Data was acquired within 48 hours of fixation on a LSRII Fortessa instrument (Becton Dickinson), stored in Listmode format, and analyzed using WinList software (Verity House Software, Topsham ME).

Enumeration of HPPs. Femurs and tibias were crushed with a sterilized mortar and pestle to generate a single cell suspension followed by erythrocyte lysis. Total live BM cells were counted by trypan blue exclusion and an equal number was plated in MethoCult GF (supports the growth of HPPs) or MethoCult with the addition of 50 ng/mL G-CSF (supports the growth of granulocyte/macrophage (G/M) progenitors [19]). The number of colonies were enumerated under a microscope on day 7 after plating and normalized to 50,000 total live BM cells for radiomitigation studies.

Measurement of cytokine response. Serum was collected after centrifugation of naturally clotted whole blood for at least 1 hour at room temperature. Serum and supernatants collected from ex vivo stimulated Nϕ were subjected to G-CSF, IL-6, and total MMP-9 ELISAs or R&D Systems Proteome Profiler Mouse XL Cytokine Array.

Statistical evaluation. Experiments were not blinded and were repeated at least once. Data are presented as mean ±SEM of biological replicates. Differences between groups within experiments were analyzed in GraphPad Prism 9 software using unpaired Student's t test. Animal survival Kaplan-Meier curves were compared using the Log-rank test. Similar variance was observed between the groups that were statistically compared. Statistical significance between groups was defined as P<0.05.

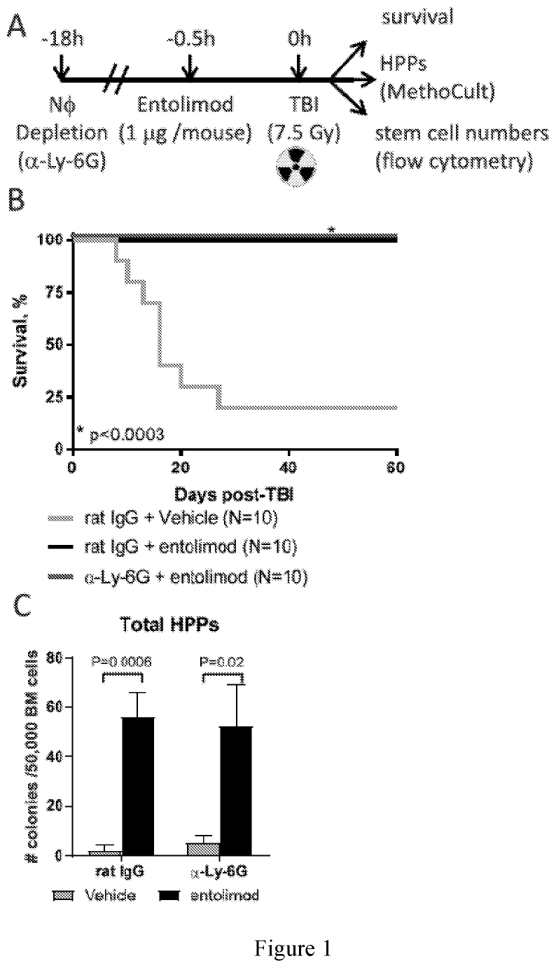

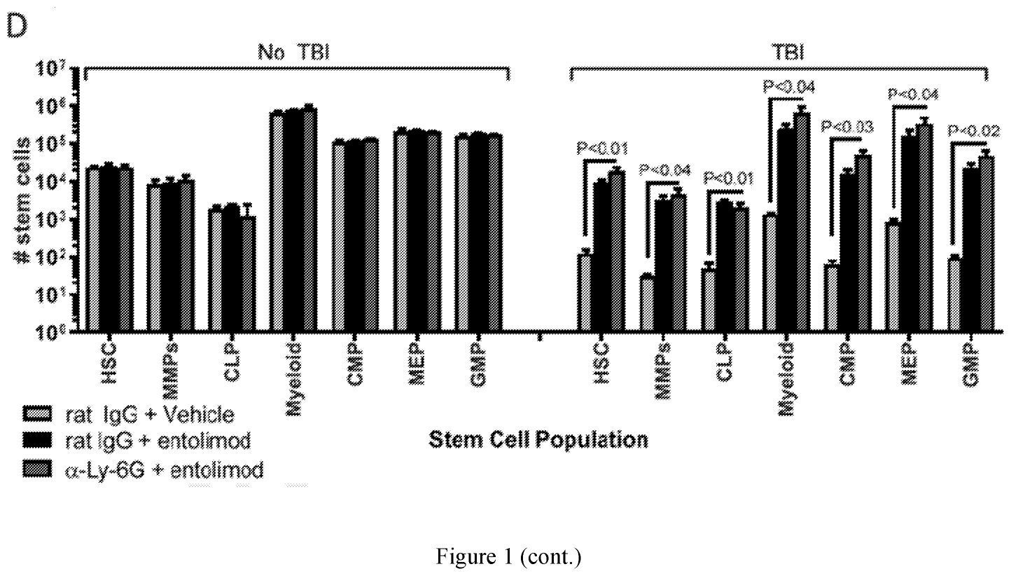

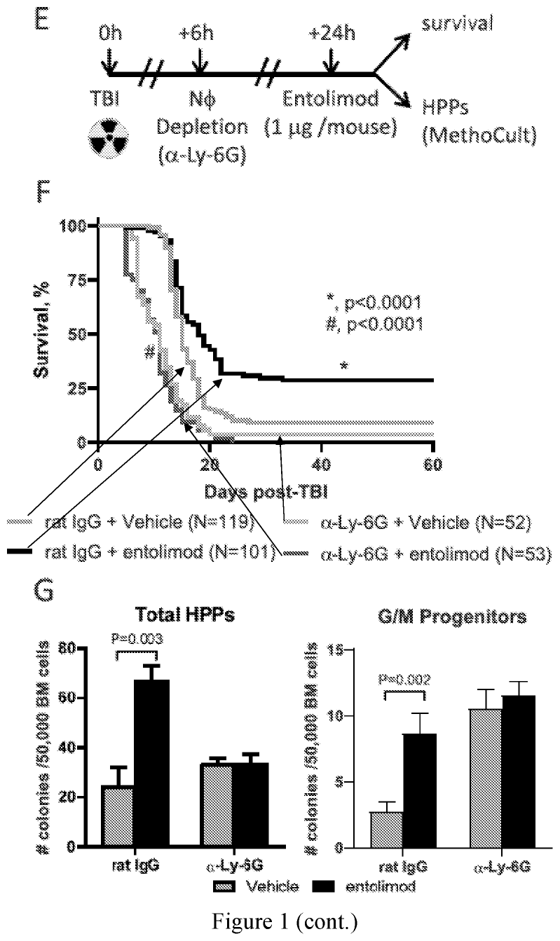

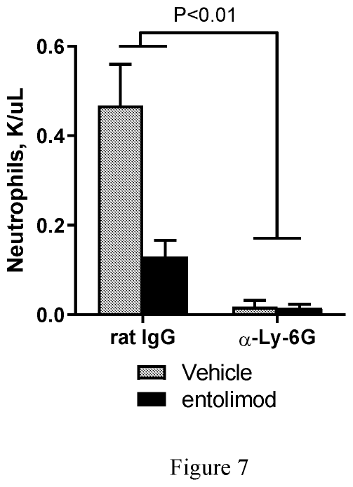

Nϕ are essential mediators of the radiomitigative but not radioprotective abilities of entolimod. Identification of Nϕ as one of three biomarkers of entolimod's efficacy as a radiation countermeasure [13] prompted us to determine whether Nϕ are cellular mediators of radioprotection and radiomitigation. We accomplished this using a loss-of-function approach to deplete Nϕ in vivo using the well-accepted α-Ly6G antibody [24], which effectively depletes Nϕ by at least 95% (FIG. 7), prior to entolimod administration in the radioprotective (FIG. 1A) and radiomitigative (FIG. 1E) schemes similarly used by us [12-16, 19].

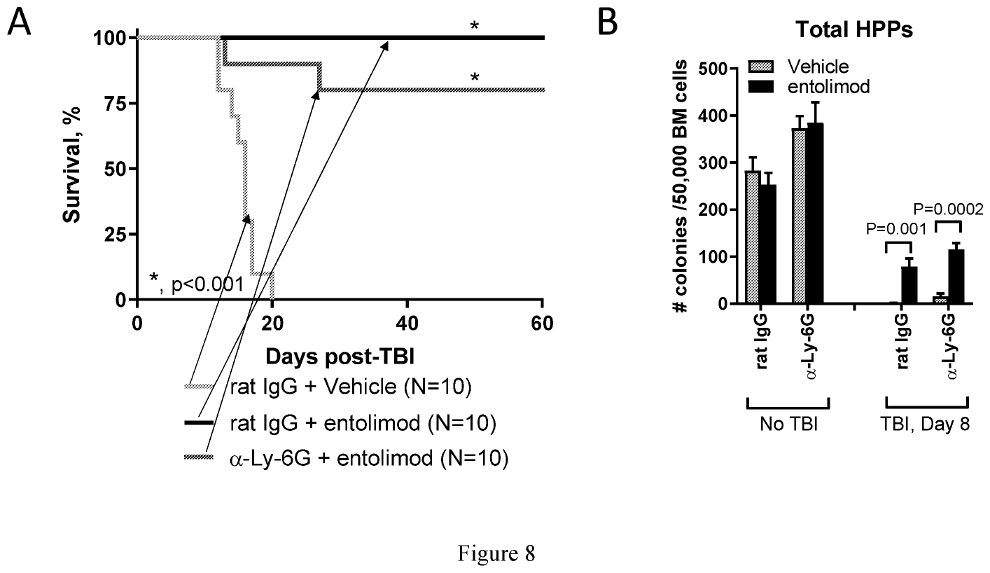

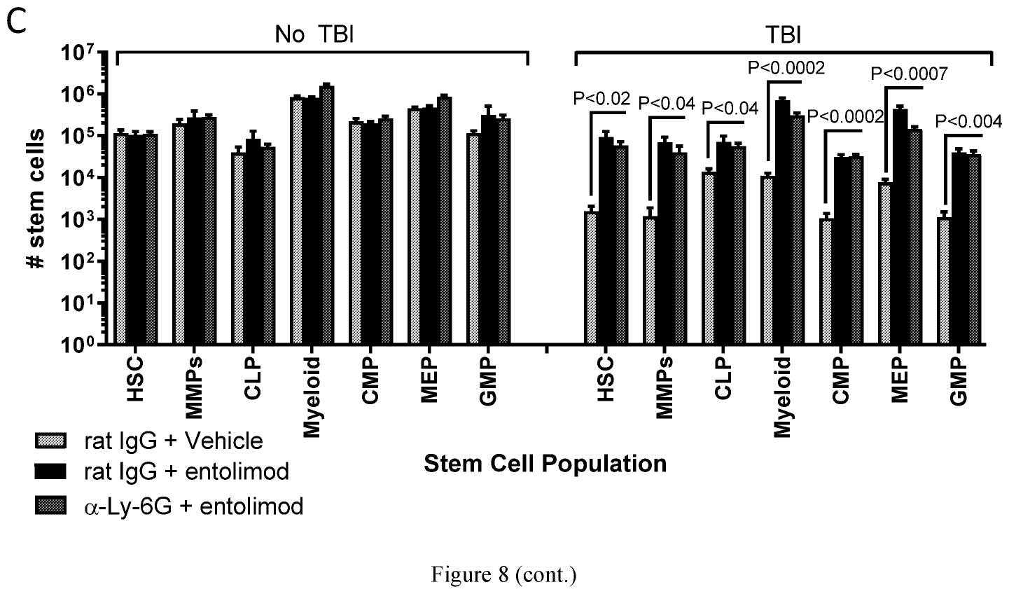

In the radioprotection scheme, entolimod protected in-bred BALB/c (FIG. 1B) and outbred NIH-S(FIG. 8A) mice from lethal ARS both in the presence (rat IgG) and absence (α-Ly6G) of Nϕ. Consistently, Nϕ depletion did not diminish the ability of entolimod to protect the number of total HPPs as measured by MethoCult in both BALB/c (FIG. 1C) and NH-S(FIG. 8B) mice. Moreover, the number of stem cell populations in the BM of BALB/c (FIG. 1D) and NIH-S(FIG. 8C) mice recovered in the presence and absence of Nϕ and was remarkably similar to stem cell numbers in control mice that did not receive TBI. Thus, the radioprotective ability of entolimod does not depend on Nϕ.

In stark contrast to radioprotection, Nϕ depletion in BALB/c mice abrogated the radiomitigative activity of entolimod (FIG. 1F). Intriguingly, Nϕ-depleted mice portrayed worse overall survival than mice with Nϕ suggesting a critical role for Nϕ in the global response of an organism to radiation damage. The absence of Nϕ also prevented the recovery of both total BM HPPs and granulocyte/macrophage (G/M) progenitors (FIG. 1G). Collectively, these data show that entolimod facilitates radioprotection and radiomitigation through distinct mechanisms, whereby, Nϕ are essential for the radiomitigative but not radioprotective activities of entolimod.

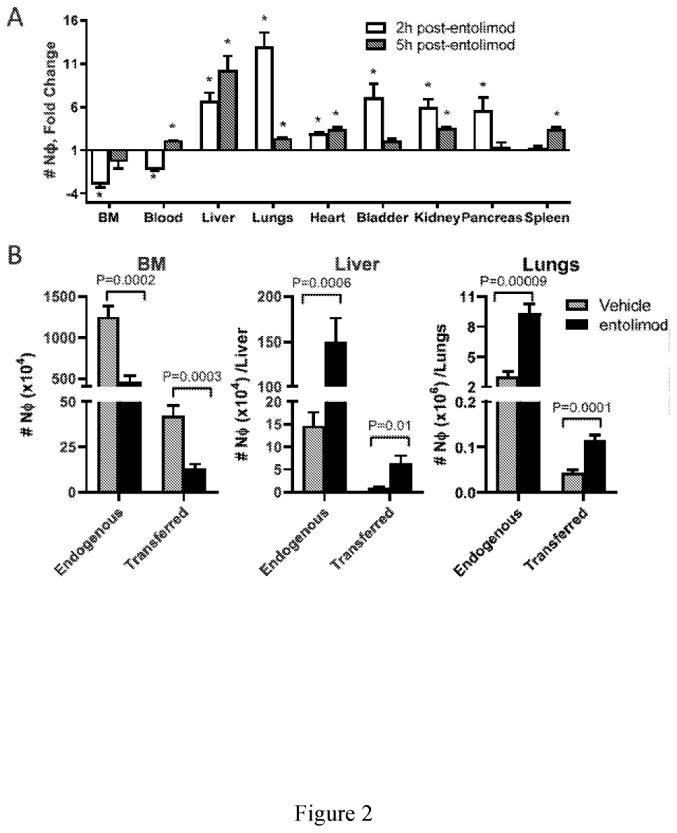

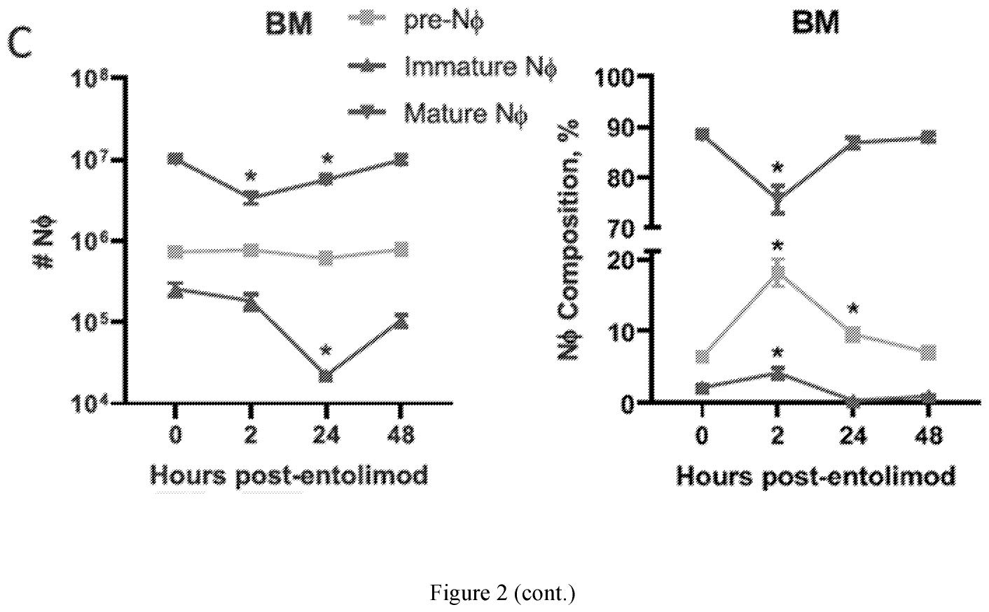

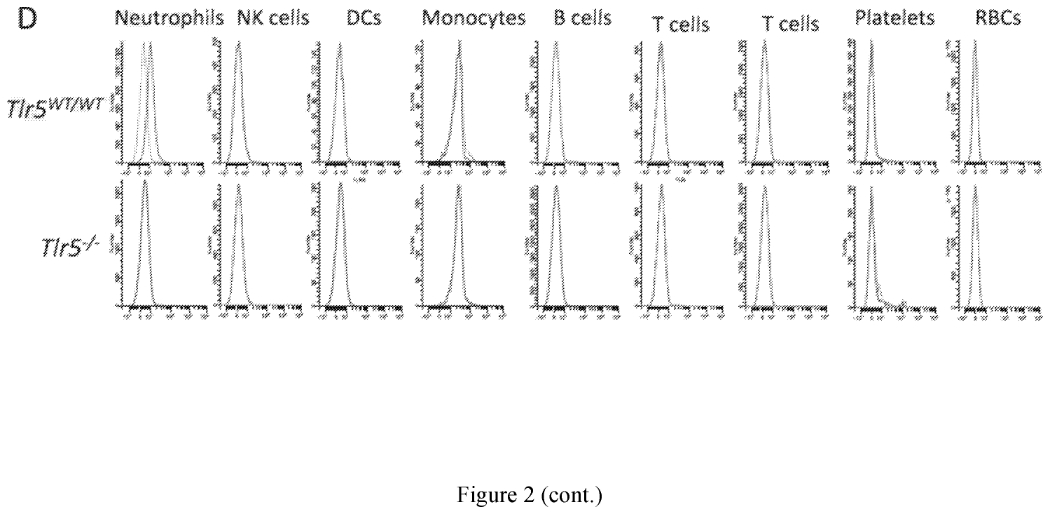

Entolimod augments Nϕ recruitment and expression of homing molecules. We next sought to perform a more comprehensive analysis of N recruitment to tissues post-entolimod, since previous work only evaluated the release of N from the BM and recruitment to the liver [19]. At 2 h post-entolimod, Nϕ exited both the BM and the blood and were also recruited to all tissues examined except for the spleen (FIG. 2A). At 5 h post-entolimod, increased Nϕ recruitment was still observed in many tissues examined (including the liver, lungs, heart, and kidneys) in addition to the blood and spleen. Adoptively transferred Nϕ were also similarly released from the BM and recruited to the liver and lungs, two of the most prominent sites of recruitment post-entolimod treatment (FIG. 2B). These results indicate that entolimod stimulates Nϕ release from the BM and recruitment to tissues through a blood-borne homing mechanism.

Since Nϕ differentiation occurs across a linear path from pre-Nϕ and then to immature and mature Nϕ [25], we sought to understand whether entolimod stimulates the release of certain Nϕ populations from the BM. Entolimod only causes a drop in the number of mature Nϕ (FIG. 2C, left), indicating a preferential release of this Nϕ population. Entolimod also changes the composition of the Nϕ population in the BM as observed by a decrease in mature Nϕ and an increase in the percentages of both pre- and immature Nϕ (FIG. 2C, right). Thus, entolimod stimulates the release of mature Nϕ from the BM which, in turn, triggers differentiation of both pre-Nϕ and immature Nϕ.

Because reliable commercially available antibodies to detect TLR5 do not exist, we developed a flow cytometry-based “sandwich” platform that utilizes biotinylated entolimod (b-entolimod) and a streptavidin-conjugated fluorochrome in combination with antibodies to identify which immune cell subsets express TLR5. Using this approach, and for additional specificity, we compared TLR5 expression in Tlr5^{WT/WT} vs. Tlr5^{-/-} mice. We found that Nϕ from the blood of wild-type mice are the only immune cells that bound b-entolimod (FIG. 2D). Importantly, Nϕ from Tlr5^{-/-} mice did not display staining, demonstrating that this approach specifically detects TLR5 expression. These results are consistent with prior observations showing that Nϕ express TLR5 [22].

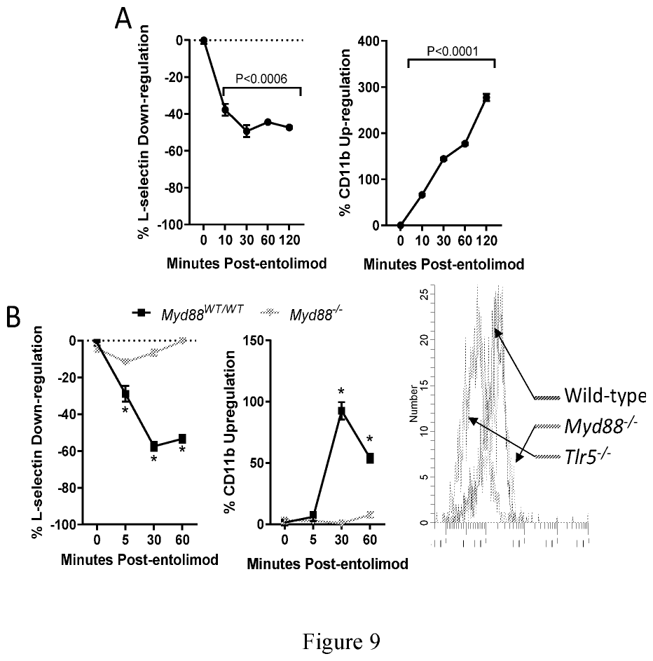

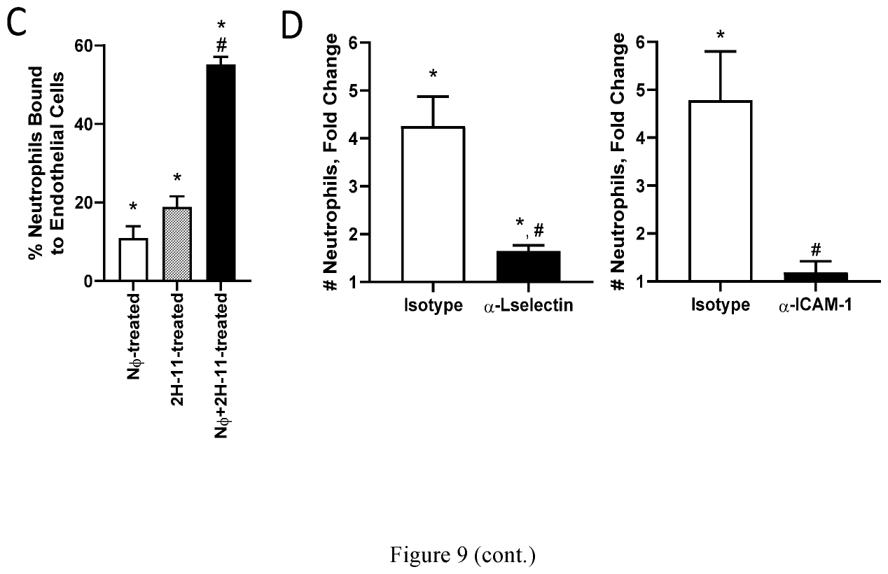

We lastly sought to determine whether TLR5 signaling through MyD88, the canonical adapter for TLR signaling [26], downregulates L-selectin and upregulates CD11b on Nϕ in order to support adhesion to the endothelium and extravasation from the blood stream into tissues [27-30]. Indeed, L-selectin was downregulated and CD11b was upregulated on Nϕ as early as 10 minutes post-entolimod and lasted for at least 120 minutes (FIG. 9A). Near complete abrogation of L-selectin downregulation and CD11b upregulation post-entolimod was observed in Myd88^{-/-} Nϕ, which was not due to a loss of cell surface expression of TLR5 on Myd8^{-/-} Nϕ (FIG. 9B) and is consistent with prior observations using a TLR4 agonist [31]. Blocking either L-selectin or ICAM-1, the ligand for CD11b, with antibodies significantly attenuated Nϕ recruitment to the liver (FIG. 9C). Lastly, entolimod-treated Nϕ had improved binding to endothelial cells and was significantly higher when both Nϕ and endothelial cells were pretreated with entolimod (FIG. 9D). Taken together, these data demonstrate that entolimod stimulates Nϕ recruitment to tissues by triggering MyD88-dependent L-selectin downregulation and CD11b upregulation.

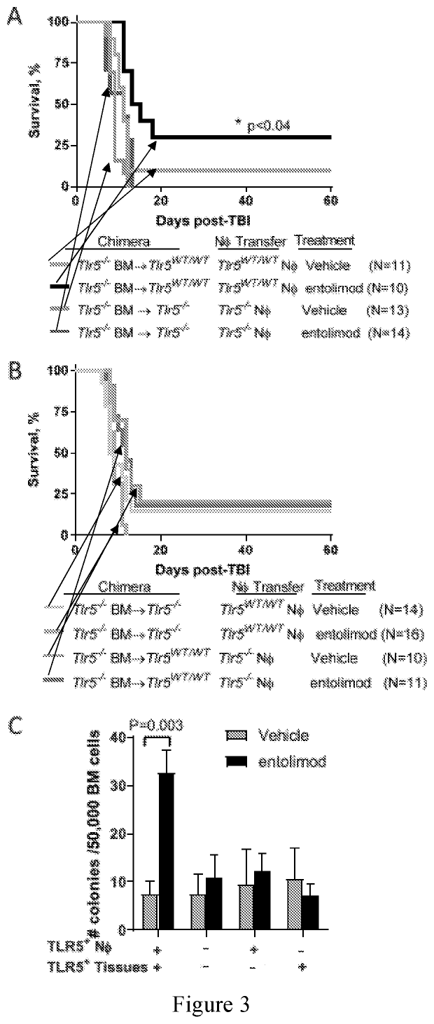

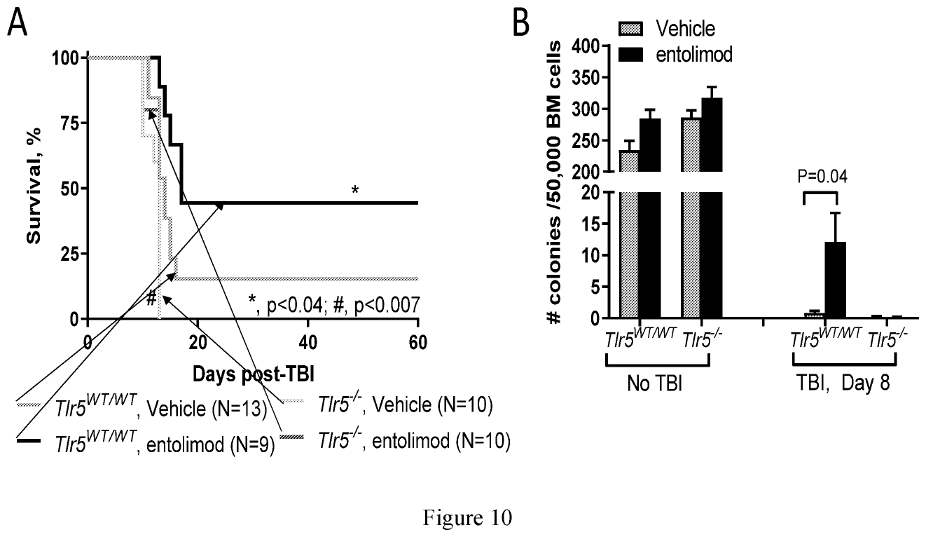

Activation of TLR5 on Nϕ and non-hematopoietic cells cooperate to mitigate radiation damage. Expression of functional TLR5 on both Nϕ (FIG. 2D) and non-hematopoietic cells (i.e., hepatocytes) [19] prompted us to determine the relative contribution of both cell populations to the radiomitigative activities of entolimod. To do this, we performed Tlr5^{WT/WT} (wild-type) or Tlr5^{-/-} Nϕ transfers in Tlr5^{-/-} bone marrow chimeras so that (i) both cell populations expressed TLR5 (wild-type Nϕ into Tlr5^{-/-} BM→wild-type chimeras); (ii) neither cell population expressed TLR5 (Tlr5^{-/-} Nϕ into Tlr5^{-/-} BM→Tlr5^{-/-} chimeras); (iii) only Nϕ expressed TLR5 (wild-type Nϕ into Tlr5^{-/-} BM→Tlr5^{-/-} chimeras); and (iv) only non-hematopoietic cells expressed TLR5 (Tlr5^{-/-} Nϕ into Tlr5^{-/-} BM→wild-type chimeras). Entolimod significantly improved overall survival when both cell populations expressed TLR5 (black vs. gray curves, FIG. 3A). The radiomitigative activity of entolimod was abrogated when both populations were TLR5-deficient (FIG. 3B), which is further substantiated by the inability of entolimod to improve survival and stimulate HPPs recovery in the BM of Tlr5^{-/-} mice post-TBI (FIG. 10). Entolimod was unable to mitigate radiation damage when either Nϕ (dark orange vs. light orange) or non-hematopoietic cells (dark green vs. light green) expressed TLR5 (FIG. 3B). Consistent with these findings, accelerated recovery of HPPs in the BM was only observed when both Nϕ and non-hematopoietic cells express TLR5 (FIG. 3C). Thus, these data show that stimulating TLR5 on both Nϕ and non-hematopoietic cells (i.e., hepatocytes) is required for mitigating lethal ARS.

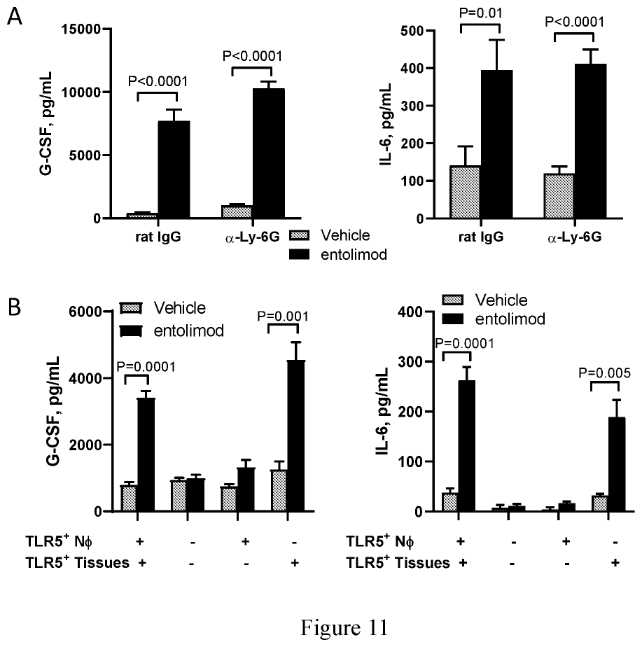

Release of MMP-9 by entolimod-stimulated Nϕ mitigates lethal radiation damage. Our prior work showing that G-CSF and IL-6 contribute to the radiomitigative activity of entolimod [14] led us to determine whether Nϕ mitigate ARS post-entolimod treatment by mediating the release of these cytokines. Depletion of Nϕ with α-Ly6G prior to entolimod treatment did not diminish expression of either G-CSF or IL-6 in serum (FIG. 11A). Moreover, entolimod stimulated comparable serum levels of both cytokines in bone marrow chimeric mice expressing TLR5 in either non-hematopoietic cells or both Nϕ and non-hematopoietic cells (FIG. 11B). Thus, the critical impact of N in the radiomitigative activities of entolimod is not mediated by the release of G-CSF and IL-6.

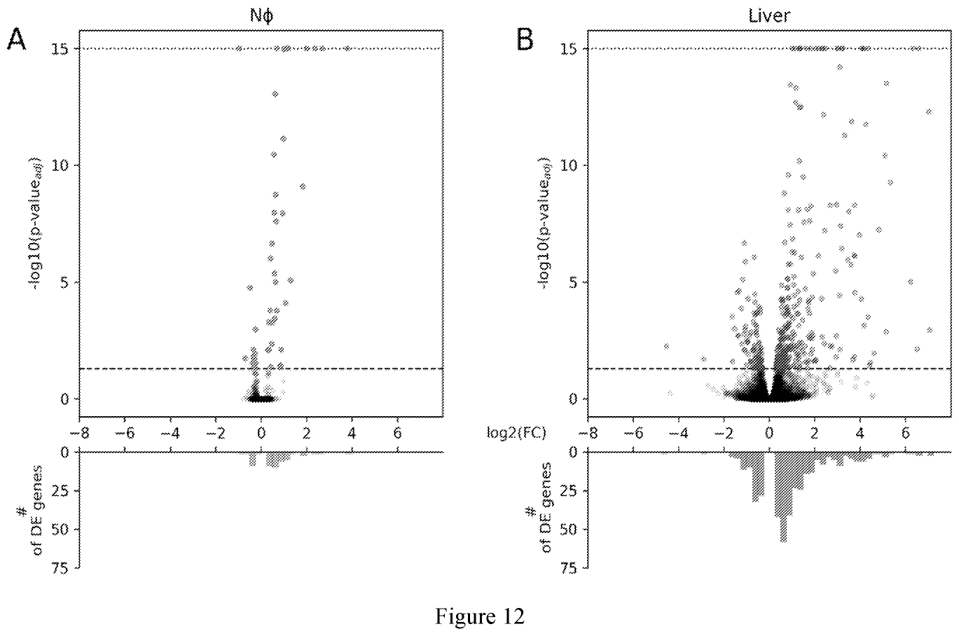

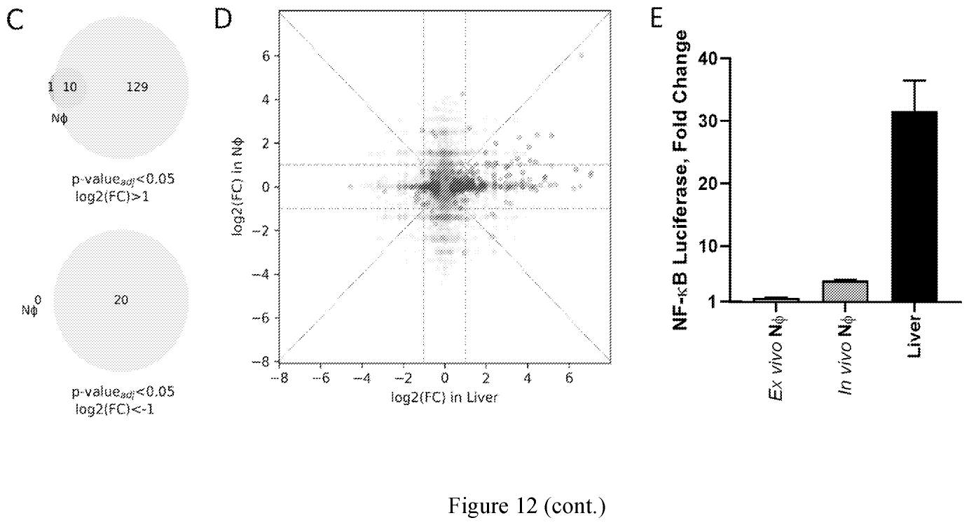

Given that Nϕ express functional TLR5 and that the underlying effects of TLR5 agonists have been associated with NF-κB-dependent transcriptional events [19], we hypothesized that changes in the transcriptional profile of Nϕ post-entolimod mitigates ARS. However, RNA sequencing data analysis showed that ex vivo stimulation of Nϕ with entolimod caused changes in transcriptional profiles that were substantially weaker—both in the numbers of responsive genes and scales of changes—as compared with that of liver cells following in vivo treatment with entolimod (FIG. 12). This observation suggests that mitigation of lethal ARS by Nϕ post-entolimod involves a transcriptional-independent mechanism.

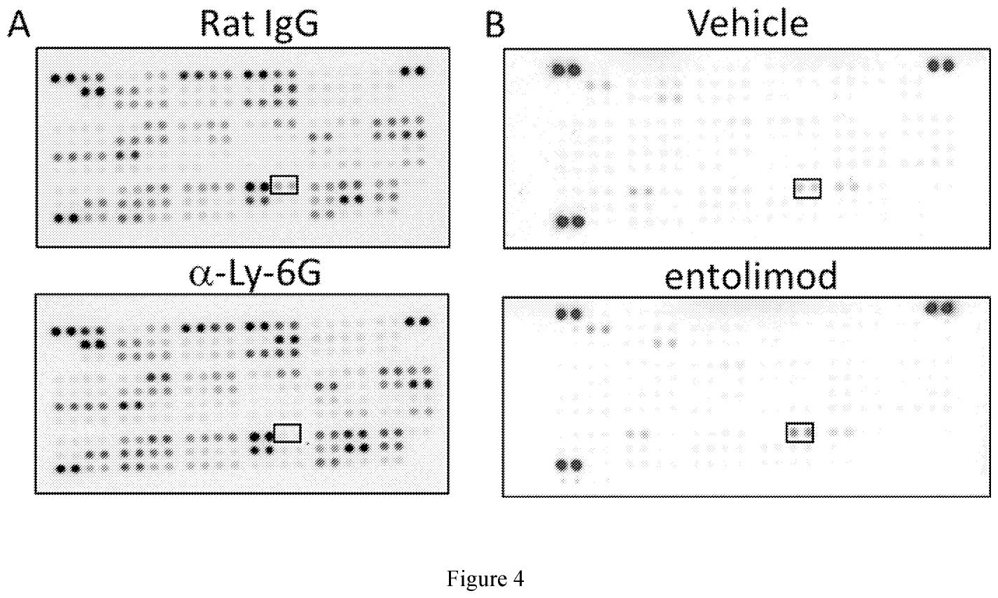

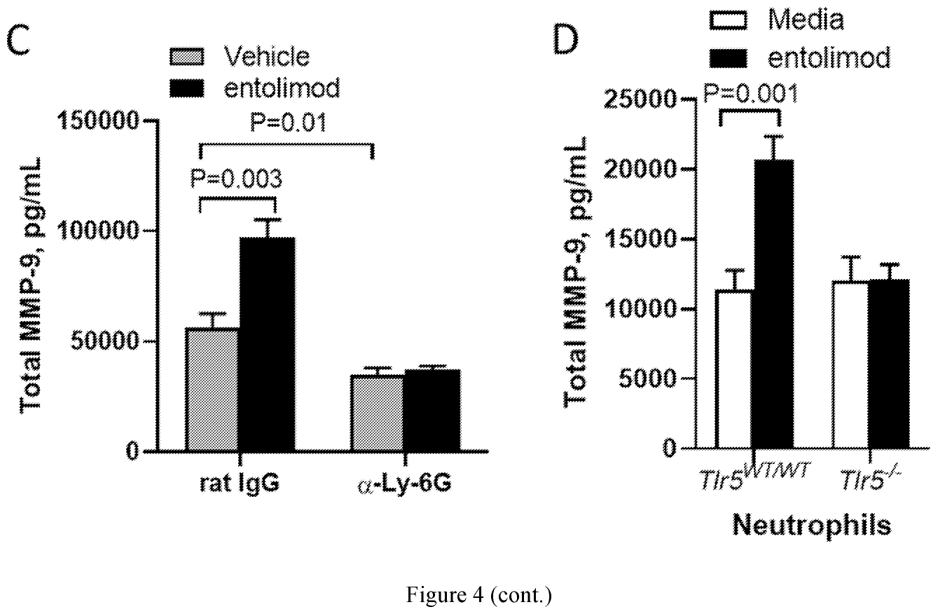

To determine potential factors released by entolimod-stimulated Nϕ that contribute to mitigation of ARS, we analyzed serum from mice that retained (rat IgG) or lacked (α-Ly6G) Nϕ using a protein microarray that simultaneously measures 111 soluble factors. We identified MMP-9 as the single factor reduced in serum of Nϕ-depleted mice when compared to control-treated serum (FIG. 4A). Consistent with this finding, MMP-9 was also the only factor released by Nϕ into supernatants after ex vivo stimulation with entolimod (FIG. 4B). In support of the protein microarray data, Nϕ depletion prior to entolimod treatment abrogated the increased levels of MMP-9 in serum as measured by ELISA (FIG. 4C). Moreover, Nϕ released MMP-9 in supernatants following ex vivo stimulation in a TLR5-dependent manner (FIG. 4D). These data show that entolimod stimulates Nϕ to release MMP-9 and are consistent with prior observations that Nϕ are the main cellular source of MMP-9 [32-36].

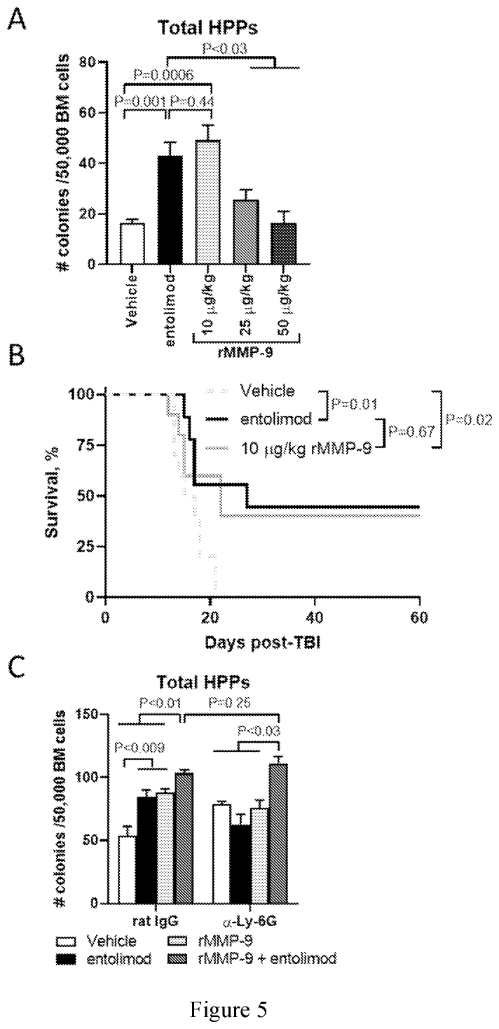

We measured whether the radiomitigative effects of rMMP-9 are linked to HPPs recovery in BM as measured by MethoCult. In fact, rMMP-9 administration 24 h post-TBI accelerated the recovery of HPPs in a dose-dependent manner with 10 μg/kg rMMP-9 showing optimal recovery similarly to entolimod (FIG. 5A). Moreover, significantly improved survival was observed following rMMP-9 administration and was strikingly similar to improved survival observed with entolimod (FIG. 5B). These data underscore that both entolimod and rMMP-9 mitigate lethal ARS.

We sought to causally link the release of MMP-9 by entolimod-stimulated Nϕ to mitigation of ARS. To do this, mice that retained (rat IgG) or lacked (α-Ly6G) Nϕ were stratified into four treatment cohorts consisting of vehicle, entolimod, rMMP-9, and rMMP-9 plus entolimod (FIG. 5C). In the presence of Nϕ, both entolimod and rMMP-9 increased the number of BM HPPs with a modest but significant increase when both rMMP-9 and entolimod were administered. In Nϕ-deficient mice, HPPs only recovered to similar levels observed in Nϕ sufficient mice when both rMMP-9 and entolimod were administered. Collectively, these data show that both the release of MMP-9 by Nϕ and the inflammatory response (e.g., G-CSF) induced by TLR5 stimulation mitigate ARS.

### DISCUSSION OF EXAMPLES

The remarkable efficacy of TLR5 agonists as a radiomitigator has been recognized, but a lack of mechanistic knowledge regarding which components of this multifunctional pathway rescue lethally irradiated organisms has significantly impeded the clinical development of TLR5 agonists as a radiation countermeasure following accidental exposure to ionizing radiation. Uncovering this mechanism is important provided the safety concerns of using bacterial protein (flagellin) derivatives, which are known to induce an IL-6-driven inflammatory response. The present disclosure provides evidence supporting the role for the release of MMP-9 by TLR5-stimulated Nϕ as an essential mediator of radiomitigation.

The disclosure includes description of differences in how the TLR5 agonist entolimod protects and mitigates radiation damage, which is reflected by the state of radiosensitive organs such as the HPS when a TLR5 agonist is administered. HPPs are intact prior to radiation thereby conferring protection from death by a series of pro-survival effects initiated by TLR5-mediated activation of NF-κB response including suppression of apoptosis (e.g., Bcl-2) and induction of natural endogenous antioxidants (e.g., superoxide dismutase) and anti-microbial factors (e.g., hepcidin) [12, 13, 19]. In stark contrast, post-irradiation treatment with radiomitigators requires rescuing an already damaged HPS that undergoes apoptotic death within the first 8 hours post-TBI [39].

The present disclosure provides data showing that administering rMMP-9 has radiomitigative capabilities to strikingly similar levels as entolimod in terms of survival and accelerating the recovery of the number of BM HPPs. Moreover, rMMP-9 plus entolimod administration restores the radiomitigative capabilities of entolimod in the absence of Nϕ, demonstrating a causal link between TLR5 agonist stimulated Nϕ and MMP-9 as a radiomitigator. The data presented in the Examples demonstrates that rMMP-9 may be useful as a radiomitigator for clinical applications involving SGS. In stark contrast to the immunogenic inflammatory response induced by entolimod that has limited its clinical development, MMP-9 is a natural endogenous human factor with no detected inflammatory activities and thus has clear advantages over entolimod as a radiomitigator. It is highly unlikely that the association of MMP-9 with tumor invasion and metastasis would have any relevance to its use as a radiomitigator since the pro-tumor effects are associated with chronic production [46] while the post-administration effects of MMP-9 as a radiomitigator would be considered shorter lasting.

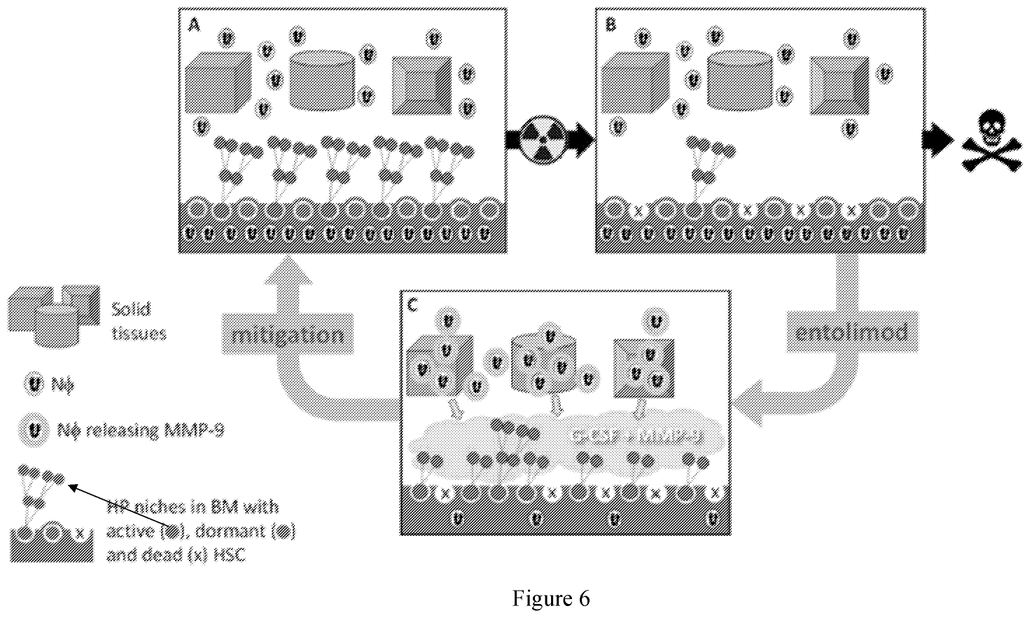

The release of MMP-9 by Nϕ plays a pivotal role in TLR5 agonist mediated regeneration of the HPS in response to radiation-induced damage (FIG. 6). However, MMP-9 may not be the sole driver of radiomitigation based on the high concentration of G-CSF and IL-6 in the circulation post-entolimod that stimulates HPS regeneration [14]. Currently, G-CSF is broadly used as an accelerator of hematopoietic restoration following myeloablative chemotherapy [47] and the G-CSF-based drugs Neupogen and Neulasta have been stockpiled as radiomitigators for emergency use [48]. Although IL-6 was shown to stimulate megakaryocytic precursor [49], its potent pyrogenic activity [50] has a controversial role as a likely mediator of flu-like syndrome in entolimod applications. Therefore, the disclosure includes use of a combination of rMMP-9 and G-CSF as a new radiomitigator lacking the drawbacks and limitations of TLR5 agonists for mass casualty situations, and other uses as further described above.

The following reference listing is not an indication that any reference is material to patentability.
1. Singh VK, Garcia M, Wise SY, Seed™. Medical countermeasures for unwanted CBRN exposures: Part I chemical and biological threats with review of recent countermeasure patents. Expert Opin Ther Pat. 2016; 26:1431-47.
2. Weiss JF, Landauer MR. Radioprotection by antioxidants. Ann N Y Acad Sci. 2000; 899:44-60.
3. Komarova EA, et al. Transgenic mice with p53-responsive lacZ: p53 activity varies dramatically during normal development and determines radiation and drug sensitivity in vivo. The EMBO journal. 1997; 16:1391-400.
4. Hendry JH, Adeeko A, Potten CS, Morris ID. P53 deficiency produces fewer regenerating spermatogenic tubules after irradiation. Int J Radiat Biol. 1996; 70:677-82.
5. Urbaschek R, Mannel DN, Urbaschek B. Tumor necrosis factor induced stimulation of granulopoiesis and radioprotection. Lymphokine Res. 1987; 6:179-86.
6. Basile LA, et al. HemaMax, a recombinant human interleukin-12, is a potent mitigator of acute radiation injury in mice and non-human primates. PloS one. 2012;7:e30434.
7. Webster GA, Perkins ND. Transcriptional cross talk between NF-kappaB and p53. Mol Cell Biol. 1999; 19:3485-95.
8. Singh VK, Newman VL, Berg AN, MacVittie TJ. Animal models for acute radiation syndrome drug discovery. Expert Opin Drug Discov. 2015; 10:497-517.
9. Waselenko JK, et al. Medical management of the acute radiation syndrome: recommendations of the Strategic National Stockpile Radiation Working Group. Ann Intern Med. 2004; 140:1037-51.
10. Dainiak N, et al. First global consensus for evidence-based management of the hematopoietic syndrome resulting from exposure to ionizing radiation. Disaster Med Public Health Prep. 2011; 5:202-12.
11. Administration USFaD. FDA approves Neupogen® for treatment of patients with radiation-induced myelosuppression following a radiological/nuclear incident.
12. Burdelya LG, et al. An agonist of toll-like receptor 5 has radioprotective activity in mouse and primate models. Science. 2008; 320:226-30.
13. Krivokrysenko VI, et al. The Toll-Like Receptor 5 Agonist Entolimod Mitigates Lethal Acute Radiation Syndrome in Non-Human Primates. PloS one. 2015;10:e0135388.
14. Krivokrysenko VI, et al. Identification of granulocyte colony-stimulating factor and interleukin-6 as candidate biomarkers of CBLB502 efficacy as a medical radiation countermeasure. J Pharmacol Exp Ther. 2012; 343:497-508.
15. Burdelya LG, et al. Toll-like receptor 5 agonist protects mice from dermatitis and oral mucositis caused by local radiation: implications for head-and-neck cancer radiotherapy. International journal of radiation oncology, biology, physics. 2012; 83:228-34.
16. Toshkov IA, et al. Mitigation of Radiation-Induced Epithelial Damage by the TLR5 Agonist Entolimod in a Mouse Model of Fractionated Head and Neck Irradiation. Radiat Res. 2017; 187:570-80.
17. Kojouharov BM, et al. Toll-like receptor-5 agonist Entolimod broadens the therapeutic window of 5-fluorouracil by reducing its toxicity to normal tissues in mice. Oncotarget. 2014; 5:802-14.
18. Fukuzawa N, Petro M, Baldwin WM, 3rd, Gudkov AV, Fairchild RL. A TLR5 agonist inhibits acute renal ischemic failure. J Immunol. 2011; 187:3831-9.
19. Burdelya LG, et al. Central role of liver in anticancer and radioprotective activities of Toll-like receptor 5 agonist. Proceedings of the National Academy of Sciences of the United States of America. 2013;110:E1857-66.
20. Haderski GJ, et al. TLR5 agonist entolimod reduces the adverse toxicity of TNF while preserving its antitumor effects. PloS one. 2020;15:e0227940.
21. Pelus LM, Bian H, King AG, Fukuda S. Neutrophil-derived MMP-9 mediates synergistic mobilization of hematopoietic stem and progenitor cells by the combination of G-CSF and the chemokines GRObeta/CXCL2 and GRObetaT/CXCL2delta4. Blood. 2004; 103:110-9.
22. Shibata T, et al. PRAT4A-dependent expression of cell surface TLR5 on neutrophils, classical monocytes and dendritic cells. International immunology. 2012; 24:613-23.
23. Shakhov AN, et al. Prevention and mitigation of acute radiation syndrome in mice by synthetic lipopeptide agonists of Toll-like receptor 2 (TLR2). PloS one. 2012;7:e33044.
24. Daley JM, Thomay AA, Connolly MD, Reichner JS, Albina JE. Use of Ly6G-specific monoclonal antibody to deplete neutrophils in mice. Journal of leukocyte biology. 2008; 83:64-70.
25. Evrard M, et al. Developmental Analysis of Bone Marrow Neutrophils Reveals Populations Specialized in Expansion, Trafficking, and Effector Functions. Immunity. 2018; 48:364-79 e8.
26. Medzhitov R, et al. MyD88 is an adaptor protein in the hToll/IL-1 receptor family signaling pathways. Mol Cell. 1998; 2:253-8.
27. Kuijpers TW, Hakkert BC, Hoogerwerf M, Leeuwenberg JF, Roos D. Role of endothelial leukocyte adhesion molecule-1 and platelet-activating factor in neutrophil adherence to IL-1-prestimulated endothelial cells. Endothelial leukocyte adhesion molecule-1-mediated CD18 activation. J Immunol. 1991; 147:1369-76.
28. Andonegui G, et al. Endothelium-derived Toll-like receptor-4 is the key molecule in LPS-induced neutrophil sequestration into lungs. The Journal of clinical investigation. 2003; 111:1011-20.
29. Simon SI, et al. L-selectin (CD62L) cross-linking signals neutrophil adhesive functions via the Mac-1 (CD11b/CD18) beta 2-integrin. J Immunol. 1995; 155:1502-14.
30. Walcheck B, et al. Neutrophil rolling altered by inhibition of L-selectin shedding in vitro. Nature. 1996; 380:720-3.
31. Hernandez A, et al. The role of MyD88- and TRIF-dependent signaling in monophosphoryl lipid A-induced expansion and recruitment of innate immunocytes. Journal of leukocyte biology. 2016; 100:1311-22.
32. Borregaard N, Cowland JB. Granules of the human neutrophilic polymorphonuclear leukocyte. Blood. 1997; 89:3503-21.
33. Borregaard N, Theilgaard-Monch K, Sorensen OE, Cowland JB. Regulation of human neutrophil granule protein expression. Curr Opin Hematol. 2001; 8:23-7.
34. Opdenakker G, et al. Gelatinase B functions as regulator and effector in leukocyte biology. Journal of leukocyte biology. 2001; 69:851-9.
35. Pugin J, et al. Human neutrophils secrete gelatinase B in vitro and in vivo in response to endotoxin and proinflammatory mediators. Am J Respir Cell Mol Biol. 1999; 20:458-64.
36. Chakrabarti S, Zee JM, Patel KD. Regulation of matrix metalloproteinase-9 (MMP-9) in TNF-stimulated neutrophils: novel pathways for tertiary granule release. Journal of leukocyte biology. 2006; 79:214-22.
37. Pruijt JF, et al. Prevention of interleukin-8-induced mobilization of hematopoietic progenitor cells in rhesus monkeys by inhibitory antibodies against the metalloproteinase gelatinase B (MMP-9). Proceedings of the National Academy of Sciences of the United States of America. 1999; 96:10863-8.
38. Heissig B, et al. Role of neutrophil-derived matrix metalloproteinase-9 in tissue regeneration. Histol Histopathol. 2010; 25:765-70.
39. Dutreix J, et al. Blood cell kinetics and total body irradiation. Radiother Oncol. 1987; 9:119-29.
40. Gialeli C, Theocharis AD, Karamanos NK. Roles of matrix metalloproteinases in cancer progression and their pharmacological targeting. FEBS J. 2011; 278:16-27.
41. Heissig B, et al. Recruitment of stem and progenitor cells from the bone marrow niche requires MMP-9 mediated release of kit-ligand. Cell. 2002; 109:625-37.
42. Vu TH, Werb Z. Matrix metalloproteinases: effectors of development and normal physiology. Genes & development. 2000; 14:2123-33.
43. Hoggatt J, et al. Rapid Mobilization Reveals a Highly Engraftable Hematopoietic Stem Cell. Cell. 2018; 172:191-204 e10.
44. Cottler-Fox MH, et al. Stem cell mobilization. Hematology Am Soc Hematol Educ Program. 2003:419-37.
45. Klein G, Schmal O, Aicher WK. Matrix metalloproteinases in stem cell mobilization. Matrix Biol. 2015;44-46:175-83.
46. Huang H. Matrix Metalloproteinase-9 (MMP-9) as a Cancer Biomarker and MMP-9 Biosensors: Recent Advances. Sensors (Basel). 2018;18.
47. Anderlini P, Champlin R. Use of filgrastim for stem cell mobilisation and transplantation in high-dose cancer chemotherapy. Drugs. 2002;62 Suppl 1:79-88.
48. Singh VK, Romaine PL, Newman VL. Biologics as countermeasures for acute radiation syndrome: where are we now? Expert opinion on biological therapy. 2015; 15:465-71.
49. Williams N, Bertoncello I, Jackson H, Arnold J, Kavnoudias H. The role of interleukin 6 in megakaryocyte formation, megakaryocyte development and platelet production. Ciba Found Symp. 1992; 167:160-70; discussion 70-3.
50. Weber J, et al. Phase I trial of subcutaneous interleukin-6 in patients with advanced malignancies. Journal of clinical oncology: official journal of the American Society of Clinical Oncology. 1993; 11:499-506.

### Material and Methods

Mice. Pathogen-free mice deficient for MyD88 (Myd88^{-/-}) on C57BL/6 background were a generous gift from Dr. Sandra Gollnick at Roswell. BALB/c-Tg (IκB α-luc)Xen reporter mice (aka NF-κB luciferase mice) similarly used by us [1] were originally purchased from Xenogen Corp. and bred in our colony at Roswell. Adult female mice between the ages of 11 and 12 weeks were assigned randomly to groups; group sizes were selected based on prior experience. No animals were excluded from further analysis in the reported studies.

Cell Lines. The authenticated mouse endothelial cell line 2H-11 was purchased from the ATCC and cultured according to their recommendations under conditions free of mycoplasma contamination.

Reagents. We purchased Hemavet compatible kits for complete blood count (CBC) from Drew Scientific, a-L-selectin (clone MEL-14) and its isotype control (rat IgG2a, x) from BioXcell, a-ICAM-1 (clone 3E2) and its isotype control (Armenian hamster IgG1, x) from BD Biosciences, Duoset ELISA kits from R&D Systems, Vybrant CFSA SE cell tracer kit from ThermoFisher, and Bright-Glo luciferase assay system from Promega. All reagents were handled according to the manufacturer's instructions.

FACS staining and analysis. For ex vivo trafficking studies, Nϕ that were isolated and treated were then stained with a mixture of FITC L-selectin (MEL-14) and PE-Cy7 CD1 lb (M1/70) antibodies, both of which were purchased from BioLegend. For in vivo trafficking studies, single cell suspension of total liver cells was stained with Panel 2 (Nϕ response) antibodies: APC-efluor780 CD45, PE-Cy7 CD11 b, PerCPCy5.5 Ly-6C, and Ax700 Ly-6G.

Nϕ trafficking studies. For L-selectin and ICAM-1-mediated Nϕ recruitment to the liver post-entolimod, cohorts of intact mice were given blocking antibodies to L-selectin (100 pg via intraperitoneal injection) or ICAM-1 (50 pg via intravenous injection) or appropriate isotype controls 30 minutes prior to entolimod. Livers were collected from mice 2 h post-treatment with entolimod or vehicle to enumerate the number of Nϕ by FACS. For entolimod-stimulated Nϕ binding to 2H-11 endothelial cells, Nϕ were isolated from the BM of intact mice and stained with CFDA dye prior to entolimod treatment and incubation with 2H-11 cells. Intensity of CFDA was measured on a Perkin Elmer Victor plate reader as a surrogate for Nϕ binding to 2H-11 cells.

RNA-seq-based comparative transcriptomic in mouse Nϕ and livers. Following magnetic isolation from BM, Nϕ were treated with vehicle or entolimod (1 ng/mL) for 30 minutes and then washed once with PBS and flash frozen. Livers were collected at 30 minutes after treatment of mice with vehicle or entolimod (0.3 pg) and flash frozen. RNA was extracted from Nϕ and livers and RNA-seq was performed similarly as described by us [2]. Briefly, the quality of the sequencing data was assessed via FastQC (www.bioinformatics.babraham.ac.uk/projects/fastqc). Reads were aligned to the mouse reference genome (UCSC mm10/GRCm38) with STAR RNA-seq aligner [3] using annotation from the same source. Reads were counted using featureCounts [4] using the same annotation. Differential gene expression and normalized counts were calculated using DESeq2 [5]. Further DE comparison, analysis, and visualization were performed using Python programming language.

### SUPPLEMENTARY REFERENCES

1. Burdelya LG, Brackett CM, Kojouharov B, Gitlin, II, Leonova KI, Gleiberman AS, et al. Central role of liver in anticancer and radioprotective activities of Toll-like receptor 5 agonist. Proceedings of the National Academy of Sciences of the United States of America. 2013;110:E1857-66.
2. Mett V, O VK, I AB, Molodtsov I, C MB, L GB, et al. A deimmunized and pharmacologically optimized Toll-like receptor 5 agonist for therapeutic applications. Commun Biol. 2021; 4:466.
3. Dobin A, Davis CA, Schlesinger F, Drenkow J, Zaleski C, Jha S, et al. STAR: ultrafast universal RNA-seq aligner. Bioinformatics. 2013; 29:15-21.
4. Liao Y, Smyth GK, Shi W. featureCounts: an efficient general purpose program for assigning sequence reads to genomic features. Bioinformatics. 2014; 30:923-30.
5. Love MI, Huber W, Anders S. Moderated estimation of fold change and dispersion for RNA-seq data with DESeq2. Genome Biol. 2014; 15:550.

TABLE 1
Enzyme solutions and methods to digest tissues for FACS

| Tissue(s) | Enzyme Solution | Method |
|---|---|---|
| Bladder and | 1 mg/mL collagenase IV | Shaking for 1 hour |
| kidneys | and 0.02 mg/mL | at 37° C. |
| | DNase, 10 mL per tissue | |
| Lungs | 1 mg/mL collagenase IV | |
| | and 0.02 mg/mL | |
| | DNase, 7 mL per tissue | |
| Pancreas | 0.1 mg/mL collagenase IV, | |
| | 10 mL per tissue | |
| Heart | 20 mM CaCl₂, 2 mg/mL | |
| | collagenase IV, 1.2 U/mL | |
| | Dispase II, 10 mL per tissue | |
| Liver | 50 μM EGTA, 0.2 mg/mL | Perfusion followed by |
| | collagenase IV | shaking for 30 minutes |
| | | at 37° C. |
| BM | N/A | Crushed with sterilized |
| | | mortar and pestle |
| Spleens | N/A | Mechanical disruption |
| Blood | N/A | 10% heparin |

TABLE 2
Antibody Panels for FACS

| | | | Catalog |
|---|---|---|---|
| Panel | Antibodies | Vendor | Number |
| #1 (TLR5 | PerCPCy5.5 CD45 | BioLegend | 103132 |
| expression) | (clone 30-F11) | | |
| | Brilliant Violet (BV) | BioLegend | 117334 |
| | 605 CD11c | | |
| | (clone N418) | | |
| | APC-Cy7 Ly-6G | BioLegend | 127624 |
| | (clone 1A8) | | |
| | PE-Cy7 CD11b | BioLegend | 101216 |
| | (clone M1/70) | | |
| | APC F4/80 (clone BM8) | BioLegend | 123116 |
| | Alexa Fluor (Ax) 700 B220 | BioLegend | 103232 |
| | (clone RA3-6B2) | | |
| | APC Ter-119 | ThermoFisher | 17-5921-82 |
| | (clone Ter-119) | | |
| | PE-Cy7 CD41 | BioLegend | 133916 |
| | (clone MWReg30) | | |
| | Streptavidin PE | ThermoFisher | 12-4317-87 |
| #2 (Neutrophil | APC-efluor780 CD45 | ThermoFisher | 47-0451-82 |
| response) | (clone 30-F11) | | |
| | BV421 CD11b | BD | 562605 |
| | (clone M1/70) | Biosciences | |
| | Ax700 Ly-6G (clone 1A8) | BioLegend | 127622 |
| | PerCPCy5.5 Ly-6C | BioLegend | 128012 |
| | (clone HK1.4) | | |
| #3 (HSCs) | biotin lineage panel (clones | BioLegend | 133307 |
| | Ter-119, M1/70, RB6-8C5, | | |
| | 145-2C11, RA3-6B2) | | |
| | Streptavidin PE-Cy7 | BD | 557598 |
| | | Biosciences | |
| | APC CD135 (clone A2F10) | BioLegend | 135310 |
| | BUV395 CD117 | BD | 564011 |
| | (clone 2B8) | Biosciences | |
| | PE Ly6A/E (clone D7) | BD | 553108 |
| | | Biosciences | |
| | BV785 CD127 | BioLegend | 135037 |
| | (clone A7R34) | | |
| | BV421 CD34 | BioLegend | 152208 |
| | (clone SA376A4) | | |
| | BV510 CD16/32 (clone 93) | BioLegend | 101333 |
| #4 (Neutrophil | biotin lineage panel | BioLegend | 133307 |
| differentiation) | (clones Ter-110, | | |
| | 145-2C11, RA3-6B2, Sca-1) | | |
| | Streptavidin PE | ThermoFisher | 12-4317-87 |
| | Ax647 CD115 | BioLegend | 135530 |
| | (clone AFS98) | | |
| | PerCPCy5.5 Gr-1 | BioLegend | 108428 |
| | (clone RB6-8C5) | | |
| | PE-Cy7 CD11b | BioLegend | 101216 |
| | (clone M1/70) | | |
| | BV510 CXCR4 | BD | 563468 |
| | (clone 2B11/CXCR4) | Biosciences | |
| | BV421 CXCR2 | BD | 566622 |
| | (clone V48-2310) | Biosciences | |
| | Ax700 Ly-6G (clone 1A8) | BioLegend | 127622 |

1. A method comprising administering to an individual in need thereof an effective amount of Matrix Metalloproteinase (MMP)-9 or a derivative thereof to inhibit, prevent development of, or treat genotoxicity.

2. The method of claim 1, wherein the individual in need of the MMP-9 or derivative thereof is at risk of developing Acute Radiation Syndrome (ARS).

3. The method of claim 1, wherein the individual in need of the MMP-9 or derivative thereof is at risk of developing genotoxicity due to chemotherapy.

4. The method of claim 1, wherein the individual in need of the MMP-9 or derivative thereof has insufficient hematopoietic function.

5. The method of claim 1, wherein the administration of the MMP-9 stimulates proliferation of dormant hematopoietic stem cells.

6. The method of claim 5, wherein the effective amount of the MMP-9 is administered in a single dose.

7. The method of claim 1, wherein the MMP-9 is the only biologically active agent administered to the individual and is sufficient to provide a stated effect.

8. The method of claim 2, wherein the administration of the MMP-9 stimulates proliferation of dormant hematopoietic stem cells.

9. The method of claim 3, wherein the administration of the MMP-9 stimulates proliferation of dormant hematopoietic stem cells.

10. The method of claim 4, wherein the administration of the MMP-9 stimulates proliferation of dormant hematopoietic stem cells.

11. The method of claim 1, wherein the effective amount of the MMP-9 is administered in a single dose.

12. The method of claim 2, wherein the effective amount of the MMP-9 is administered in a single dose.

13. The method of claim 3, wherein the effective amount of the MMP-9 is administered in a single dose.

14. The method of claim 4, wherein the effective amount of the MMP-9 is administered in a single dose.


SEQUENCE LISTING (1 sequence)

SEQ ID NO: 1 - protein, 707 aa, synthetic construct
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED